(19) United States
(12) Patent Application Publication
(10) Pub. No.: US 2017/0364647 A1
(43) Pub. Date: Dec. 21, 2017

(54) AUTOMATED DERIVATION OF QUALITY ASSURANCE RULES

(71) Applicant: KONINKLIJKE PHILIPS N.V., EINDHOVEN (NL)

(72) Inventors: MERLIJN SEVENSTER, CHICAGO, IL (US); THOMAS ANDRE FORSBERG, HAYWARD, CA (US)

(21) Appl. No.: 15/536,813

(22) PCT Filed: Dec. 18, 2015
(86) PCT No.: PCT/IB2015/059772

§ 371 Date: Jun. 16, 2017

Related U.S. Application Data
(60) Provisional application No. 62/094,088, filed on Dec. 19, 2014.

(51) Int. Cl. G06F 19/00; G06F 17/24; G06F 17/27; G06T 7/00; A61B 5/055; A61B 8/08

(52) U.S. Cl. CPC G06F 19/345; G06F 19/3487; G06F 17/248; G06F 17/2785; G06F 19/321; A61B 5/055; A61B 8/0883; G06T 7/0012; G06T 2207/10088; G06T 2207/10136; G06T 2207/30048

(57) ABSTRACT

A system and method generates a rule set. The method being performed by a rule generating device includes receiving a plurality of previously generated reports where each of the previously generated reports includes respective analysis content of a respective image. The method includes generating a candidate rule based upon the analysis content where the candidate rule is configured to increase a quality assurance of future reports. The method includes generating a respective score for each candidate rule based upon the candidate rule and the previously generated reports. The method includes including the candidate rule into the rule set when the score is above a predetermined threshold.

An imaging device is used to visualize internal structures of a body. For example, the imaging device may use two-dimensional, three-dimensional, and/or Doppler ultrasound to create images of an internal organ such as the heart. The data gathered from using this technique may provide a basis from which an anatomical image may be generated. Specifically, a cross sectional, axial image of internal structures of the body may be represented in a two-dimensional image or more complex images may be generated as a three-dimensional image. In this manner, a non-invasive, no-dose modality for imaging soft tissue is provided. The image may be used by a user such as a physician, technician, etc., to determine whether the internal structures captured in the image are healthy, injured, etc., by determining whether any anomalies are present.

By viewing the generated image of the internal organ, the user may analyze a condition of the organ. The user may be provided a user interface in which an organ and subcomponents thereof may have different pre-defined finding codes (FC) associated therewith that indicates a condition as witnessed through the image. The FC consists of a code component (e.g., LV800.1) and a textual component (e.g., “Left ventricle is normal”) such that a narrative report may be generated based upon any entered FCs. Specifically, during reporting, a user such as a cardiologist when related to echocardiograms selects FCs from a drop-down menu which then appears as selectable items in a reporting pane. Upon finalization of the report, the narrative report is created that consists of the textual components of the entered FCs.

Given this format in which reports may be generated, quality assurance rules may be required, particularly in echocardiogram interpretation workflows. For example, a user may enter FCs that are contradictory in nature such as a first entered FC indicating a left ventricle is normal while a second entered FC indicates that the left ventricle is severely dilated. Indeed, one skilled in the art may read the report and be unable to properly conclude the condition of the organ based upon this contradictory result. However, drafting a reliable rule set for a particular institution or in a general manner is labor and knowledge intensive. Furthermore, not every clinical site may have the expert resources available to develop a satisfactory rule set in house. Due to localization of structured report content, no one rule set may be developed that is shared between multiple clinical sites.

Accordingly, it is desirable to dynamically generate a set of rules that are related to a particular institution based upon the manner in which the institution itself operates in handling reports.

The exemplary embodiments relate to a system and method for generating a rule set. The method comprises receiving, by a rule generating device, a plurality of previously generated reports, each of the previously generated reports including respective analysis content of a respective image; generating, by the rule generating device, a candidate rule based upon the analysis content, the candidate rule configured to increase a quality assurance of future reports; generating, by the rule generating device, a respective score for each candidate rule based upon the candidate rule and the previously generated reports; and including, by the rule generating device, the candidate rule into the rule set when the score is above a predetermined threshold.

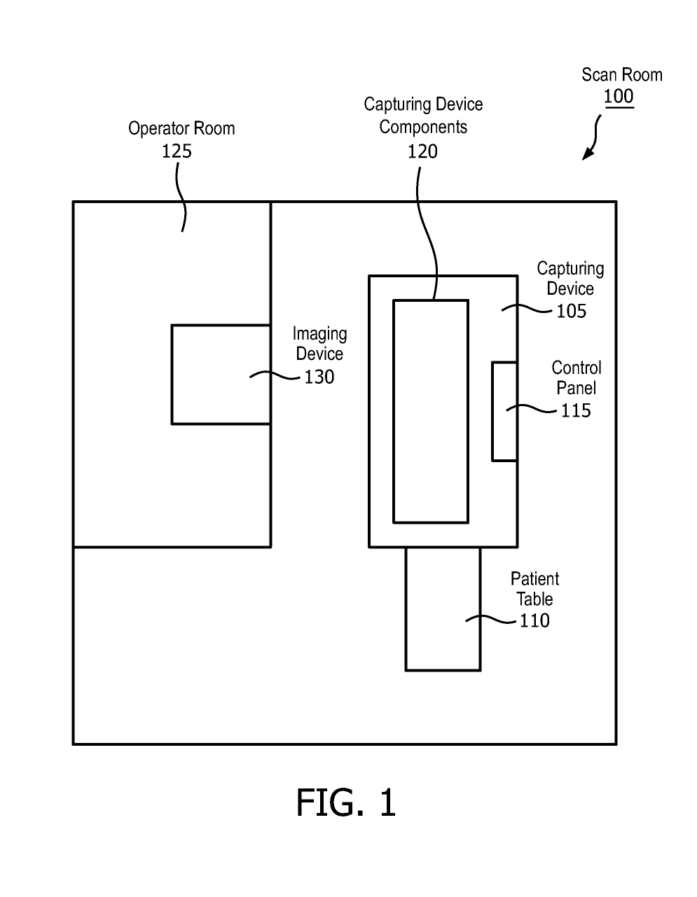

FIG. 1 shows a system for a scan room according to the exemplary embodiments.

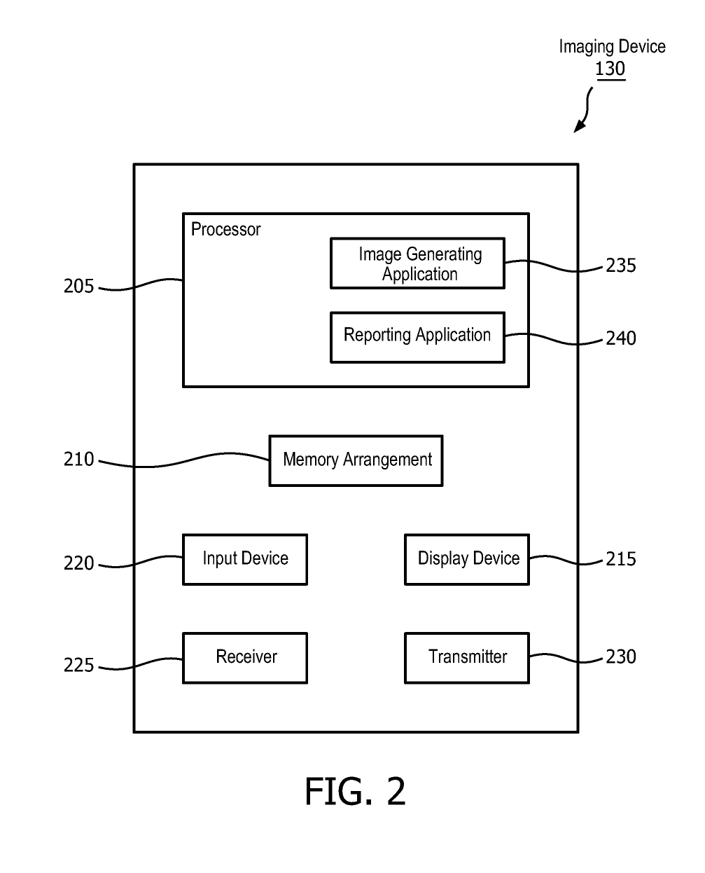

FIG. 2 shows an imaging device according to the exemplary embodiments.

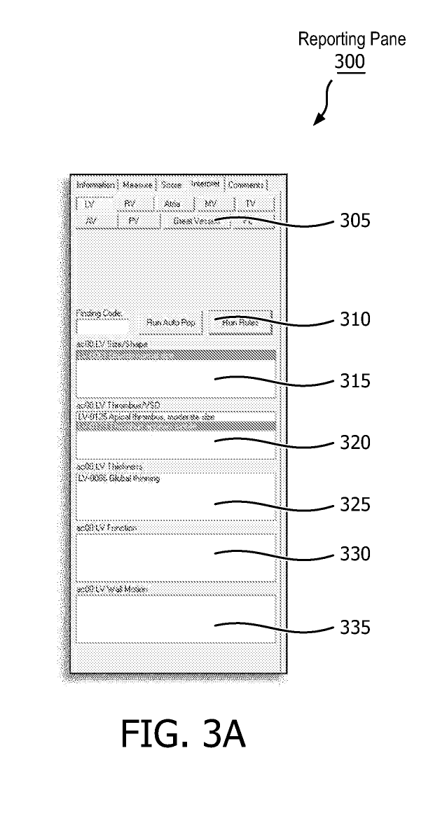

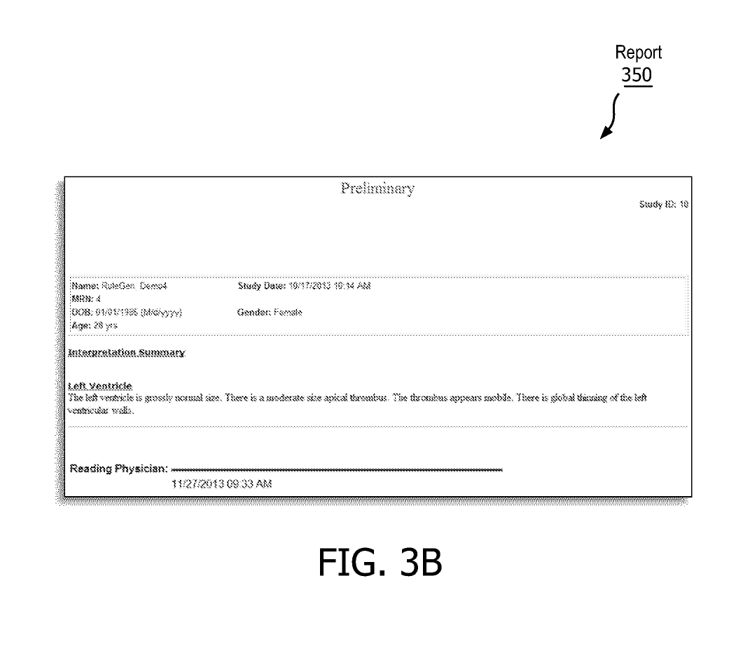

FIG. 3A shows a reporting pane used to include finding codes according to the exemplary embodiments.

FIG. 3B shows a report generated based upon the finding codes entered in the reporting pane according to the exemplary embodiments.

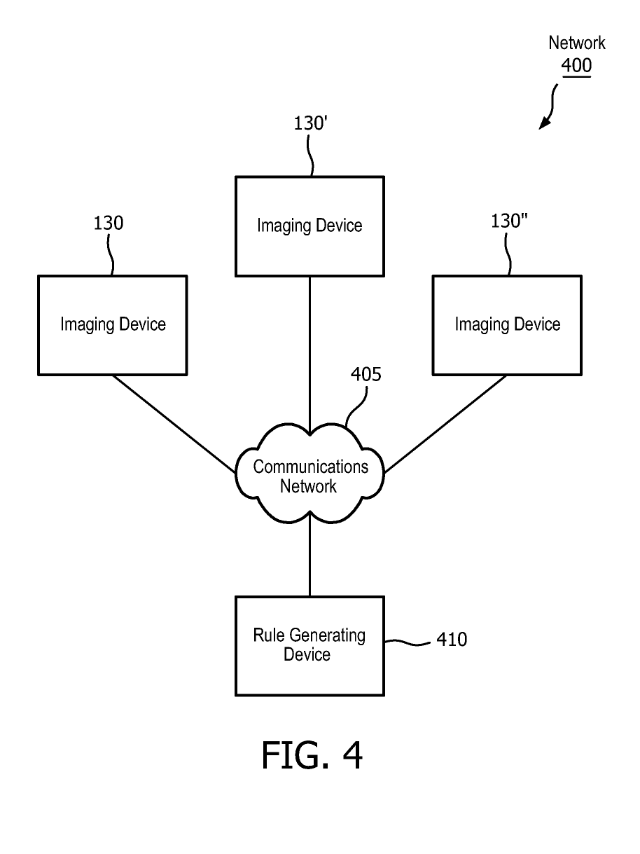

FIG. 4 shows a network for a plurality of imaging devices to communicate with a rule generating device according to the exemplary embodiments.

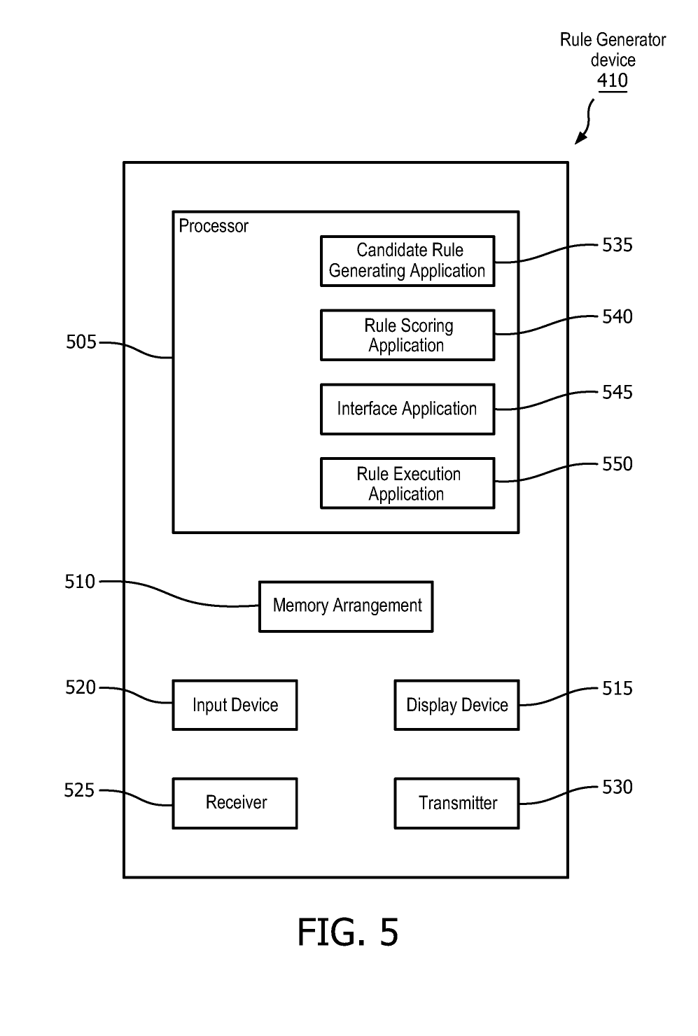

FIG. 5 shows a rule generating device according to the exemplary embodiments.

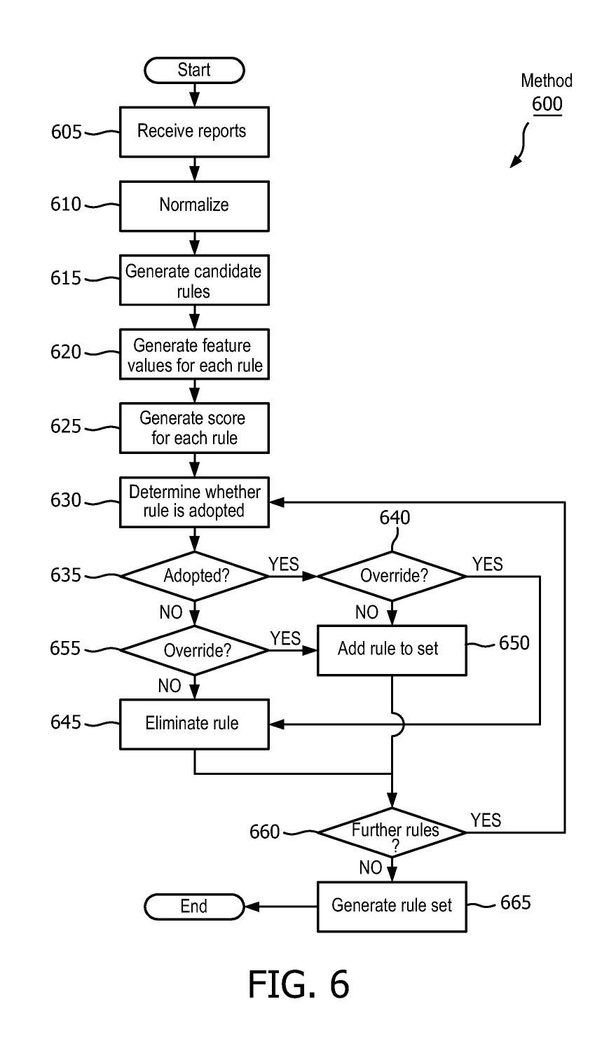

FIG. 6 shows a method of generating a rule set according to the exemplary embodiments.

The exemplary embodiments may be further understood with reference to the following description of the exemplary embodiments and the related appended drawings, wherein like elements are provided with the same reference numerals. The exemplary embodiments are related to a system and method of generating a rule set for a system utilizing a plurality of finding codes (FC). Specifically, the rule set indicates an interaction between the various FCs such as a first FC being contrary to a second FC so that when appearing together in a single report, an action such as an alert may be performed. Accordingly, a report that includes the results of the included FCs are correct and do not contradict one another. The rule set, the FCs, the report, the alert, and a related method will be explained in further detail below.

The exemplary embodiments are described herein with regard to an imaging device and a plurality of FCs used in conjunction with an analysis of images generated by the imaging device by a user such as a technician. However, it should be noted that the use of an imaging device and the accompanying manner of image analysis is only exemplary. Those skilled in the art will understand that the exemplary embodiments may be applied to any system that utilizes descriptors such as FCs that identify a characteristic. Furthermore, the use of FCs is also only exemplary. Specifically, the FCs may represent any identification of a characteristic within an object that is entered by a user. Therefore, the imaging device may represent any system in which the exemplary embodiments may be utilized, the FCs may represent any identifier used within the system in which exemplary embodiments may be utilized, and the analysis of images may represent any process in which a user provides a plurality of inputs that are evaluated in which the exemplary embodiments may be utilized.

FIG. 1 shows a system for a scan room 100 according to the exemplary embodiments. The scan room 100 is used for a patient who requires an imaging to be performed. For example, the patient may require a magnetic resonance imaging (MRI) image to be generated by performing a capturing procedure on a specific body portion. In another example, an echocardiogram using ultrasound may be used to generate an image also by performing a capturing procedure on a specific body portion. The scan room 100 includes a capturing device 105 which has a patient table 110, a control panel 115, and capturing device components 120 as well as an operator room 125 including an imaging device 130.

According to the exemplary embodiments, the capturing device 105 may perform a capturing procedure such as a scan in which data is gathered from the corresponding mechanism of the capturing procedure and transmitted to the imaging device 130. When the capturing device 105 is a MRI device, the capturing procedure may be performed by having a patient lie on the patient table 110 and utilize the capturing device components 120 to perform the scan. The patient may be moved within a bore of the capturing device 105 via inputs received on the control panel 115. The control panel 115 may allow an operator to move the patient table 110 for an alignment to be performed where the patient table 110 is moved to the isocenter (the point in space through which the central beam of radiation is to pass).

With particular reference to the capturing device 105 being an echocardiogram device, the echocardiogram device is configured to generate information associated with generating an image based upon a sonogram of the heart. Accordingly, the echocardiogram device uses two-dimensional, three-dimensional, and Doppler ultrasound to create images of the heart. There are many different configurations in which the echocardiogram procedure may be performed. In a first configuration, the echocardiogram device may utilize a transthoracic echocardiogram or cardiac ultrasound in which a transducer or probe is placed on a chest wall or thorax of the patient and images are taken therethrough. In a second configuration, a transesophageal echocardiogram procedure may be performed in which a specialized probe including an ultrasound transducer is passed into the esophagus of the patient to allow image and Doppler evaluation from a location directly behind the heart. In other configurations, a stress echocardiogram procedure (imaging while the heart is under physical stress) or three-dimensional echocardiogram procedure (imaging to produce moving images over time through an appropriate processing system) may be performed. Regardless of the configuration being used, information is gathered relating to the heart and is recorded and transmitted from the capturing device 105 to the imaging device 130 to construct an image of the scanned area of the body.

When the capturing device 105 is an echocardiogram device, the capturing device components 120 may include the probe which includes ultrasound components such as ultrasound coils or crystals. Accordingly, the ultrasound component may generate ultrasound waves that propagate from the probe toward the heart. The ultrasound waves may reflect off tissue and return toward the probe or other component. The return waves may be measured by a receiver (e.g., housed in the probe or the other component). This information may be processed and transmitted to the imaging device 130. For example, the capturing device components 120 may also include a short or long range transmitter in a wired or wireless manner to transmit the information.

Using the above capturing procedure, the imaging device 130 may be capable of generating the image. FIG. 2 shows the imaging device 130 of FIG. 1 according to the exemplary embodiments. The imaging device 130 may be configured to communicate using a wired or wireless arrangement with the capturing device 105. Accordingly, the imaging device 130 may include a receiver 225 and a transmitter 230 that may include the corresponding communication arrangement. However, it should be noted that the imaging device 130 may include a combined transceiver to provide the functionalities of the receiver 225 and the transmitter 230. The receiver 225 and the transmitter 230 may be for a short range wireless communication (with the capturing device 105 within a predetermined range) or for long range wireless communications such as with a network.

The imaging device 130 may include a processor 205 and a memory arrangement 210. The processor 205 may execute an image generating application 235 that processes the ultrasound return signal information provided by the capturing device 105 to generate an image to be viewed by the user. As will be described in further detail below, while the user is viewing the image generated by the imaging device 130, a condition of areas in the heart may be indicated using various indicators such as FCs. The processor 205 may further execute a reporting application 240 that generates a report based upon the entered indicators during an analysis of the image. As will be described in further detail below, the reporting application 240 may further be configured to process the entered indicators to substantially eliminate any contradictions that may ultimately be present on the report. As will also be described in further detail below, this may be based upon criteria determined for a particular institution utilizing the capturing device 105 and the imaging device 130. The applications 235, 240, the indicators, and the criteria may be stored in the memory arrangement 210.

The imaging device 130 may also include a display device 215 and an input device 220. For example, the processor 205 may execute the image generating application 235 that utilizes the data received from the capturing device 105 (via the receiver 225) to generate the images of the scan. These images may be shown on the display device 215. The images may also be shown one at a time or multiple images concurrently. The image generating application 235 and/or a user interface that is also shown on the display device 215 may provide the user with a selection in the manner of how the images are to be shown as well as a layout for when multiple images are shown. The input device 220 may receive inputs from the operator to control operation of the capturing device components 120 to select a slice to be scanned for the image to be generated. The input device 220 may also enable indicators to be entered during an analysis of the images.

The exemplary embodiments relate to generating a rule set applicable to processing entered indicators during an analysis of at least one image shown from performing an imaging procedure. In one example, the image may be of a heart during an echocardiogram procedure of a patient. The rule set may also be defined for a given device, a given set of devices such as within an institution or department, a given region that has a common practice of analyzing images, etc. The rule set may also be defined as a broad, generic set used by any user utilizing the capturing device 105 and the imaging device 130. However, those skilled in the art will understand that there are differences in the analysis of images, particularly from institution to institution. As will be described herein, the rule set may be generated based upon analyses performed in a given institution including a plurality of capturing devices 105 and imaging devices 130. Specifically, the rule set may be based upon indicators entered during the analysis in a reporting pane used to ultimately generate a report.

FIG. 3A shows a reporting pane 300 used to include FCs according to the exemplary embodiments. While a patient is having the imaging procedure performed, the user may view the generated images. While viewing the images, the user may utilize a user interface such as the reporting pane 300. The reporting pane 300 enables the user to include the various findings from analyzing the images. Specifically, the reporting pane may be FC driven in which the findings are entered as indications based upon FCs including a code component and text component.

As illustrated in the exemplary reporting pane 300, a plurality of heart area tabs 305 may be shown. The user may select one of these heart area tabs 305 to define the subsequent portions of the reporting pane 300. For example, the heart area tabs 305 may include the left and right ventricles, the atria, the different valves, etc. As shown, the user may have selected the heart area tab 305 corresponding to the left ventricle. It is again noted that the use of the echocardiogram is only exemplary, therefore the use of the heart as the body part of interest is only exemplary. Those skilled in the art will understand that the reporting pane 300 may be for any body part or may represent any subcomponent in an overall system that is to be analyzed.

Once the heart area tab 305 is selected, the user may begin to enter the indications as FCs in the input area 310. The input area 310 may include different manners of entering the FCs. For example, an input box may enable a user to manually enter the desired FC. In another example, a menu may be provided in which the user may select one or more FCs. The menu may be accessed through a variety of means such as a pull down menu (e.g., incorporated with the input box), a pop-up window, etc. Once the FCs are entered, a corresponding section may list the FC therein in which the corresponding section relates to a characteristic of the selected heart portion. For example, a plurality of windows 315-335 may be shown where each window 315-335 relates to a respective characteristic of the selected heart part (e.g., size/shape, thrombus, thickness, function, wall motion, etc.). As shown, four FCs may have been entered in which a first FC corresponds to a size or shape of the left ventricle; second and third FCs correspond to a thrombus of the left ventricle, and a fourth FC corresponds to a thickness of the left ventricle.

Once the reporting pane 300 is done being used, the imaging device 130 may process the findings entered as FCs and generate a report. FIG. 3B shows an exemplary report 350 generated based upon the FCs entered in the reporting pane 300 of FIG. 3A according to the exemplary embodiments. The report 350 may be a result of using the reporting pane 300. With respect to the specific FCs entered in the reporting pane 300 of FIG. 3A, the report 350 includes the findings in a textual format for ease of reading. The report 350 may also include information of the patient, information of when the imaging procedure was performed, information of when the analysis of the generated images was performed, information (and/or signature) of the user or technician performing the analysis, etc.

It should be noted that the imaging device 130 and the processor 205 may also execute an analyzing application that is configured to perform the analysis of the generated images in an automated manner. The analyzing application may be pre-configured to determine the various findings and enter the FCs in a substantially similar manner as when the user manually performs the analysis. However, it should be noted that this automated process may also include a manual component in which the user may perform a secondary check of the automated analysis to verify the findings of the analyzing application.

As discussed above, the report 350 may include at least one contradiction based upon the entered FCs. For example, while viewing a first image in a set of images generated for the heart of the patient, the user may view that the left ventricle is normal. Accordingly, the user may enter the FC corresponding to this characteristic. However, while viewing a second image in the set of images, the user may view that the left ventricle is severely dilated. Accordingly, the user may enter the FC corresponding to this characteristic. The resulting report from these entered FCs will therefore include a contradiction that states the left ventricle is both normal and dilated. When a subsequent reader of the report views this, the reader is incapable of understanding whether the left ventricle is either normal or dilated. In view of these scenarios forming, the exemplary embodiments are configured to recognize when these contradictions exist and perform an appropriate action such as providing an alert or removing one or more FCs that would eliminate the contradiction.

Although a manufacturer of the imaging device 130 may be capable of generating a rudimentary rule set for use upon first performing an imaging procedure and analysis of the generated images, those skilled in the art will appreciate that rule sets may not reasonably be transferred between institutions or departments if the FC bases are not aligned. However, the FC-driven quality assurance of the reports are also of note as high-quality reporting is increasingly important in healthcare enterprises. Therefore, the rule set that would apply to more than basic contradictions may not be made and provided. Drafting the rule set for the institution is often labor intensive and knowledge intensive and the institution may not be capable or have the resources to properly define the rule set. Therefore, the exemplary embodiments provide an automated mechanism to derive quality assurance rules based on a set of retrospective reports and assess usefulness in an offline or online workflow to overcome several disadvantages such as non-portability of rule sets and lack of resources to manually create rule sets.

FIG. 4 shows a network 400 for a plurality of imaging devices 130, 130′, 130″ to communicate with a rule generating device 410 via a communications network 405 according to the exemplary embodiments. As discussed above, the exemplary embodiments may be configured to generate the rule set based upon previously generated reports as well as actions taken subsequently to the reports such as corrections that were made.

The imaging device 130 was discussed above. The imaging devices 130′ and 130″ may represent imaging devices that are substantially similar to the imaging device 130. For example, the imaging devices 130, 130′, 130″ may relate to echocardiogram procedures. However, it should be noted that the imaging devices 130, 130′, 130″ may also be for different imaging procedures such as MRI and the imaging devices 130, 130′, 130″ may each perform respective imaging procedures.

The communications network 405 may be used to assist in communication between the imaging devices 130, 130′, 130″ and the rule generating device 410. According to the exemplary embodiments, the communications network 405 may be a network environment using logical connections to one or more remote computers having processors. The logical connections may include, for example, a local area network (LAN) and a wide area network (WAN) that utilize a wide variety of different communication protocols. Those skilled in the art would appreciate that such network computing environments typically encompass many types of computer systems configurations, including personal computers, hand-held devices, multi-processor systems, microprocessor-based or programmable consumer electronics, network PCs, minicomputers, mainframe computers, etc.

The rule generating device 410 may be a component that automatically generates the rule set to be used by the institution including the imaging devices 130, 130′, 130″. FIG. 5 shows the rule generating device 410 according to the exemplary embodiments. The rule generating device 410 may be configured to communicate using a wired or wireless arrangement with the communications network 405. Accordingly, the rule generating device 410 may include a receiver 525 and a transmitter 530 that may include the corresponding communication arrangement. However, like the imaging device 130, it should be noted that the rule generating device 410 may include a combined transceiver and the communications may be for a short range wireless communication or for long range wireless communications.

The rule generating device 410 may include a processor 505 and a memory arrangement 510. The processor 505 may execute a plurality of different applications such as a candidate rule generating application 535, a rule scoring application 540, an interface application 545, and a rule execution application 550. As will be described in further detail below, the candidate rule generating application 535 may create candidate rules based on pre-existing logical templates and a FC vocabulary used in a database of structured reports; the rule scoring application 540 may assess a candidate rule and rate it according to a predetermined scale to determine an adoption thereof; and the interface application 545 may expose the adopted rules to external agents such as the rule execution application 550. The applications 535-550 may be stored in the memory arrangement 210.

The rule generating device 410 may also include a display device 515 and an input device 520. For example, the processor 505 may provide a user interface for a user to analyze a candidate rule set generated by the rule generating device 410. The input device 520 may receive inputs from the user to manipulate the candidate rules and candidate rule sets. The input device 520 may also represent a component that enables reports to be received by the rule generating device 410. In a first example, a user may manually enter the previous reports via the input device 520. In a second example, the input device 520 with the receiver 525 may receive the previous reports.

Initially, the rule generating device 410 may compile a plurality of previous reports. For example, a database of structured reports may be received from a particular institution that utilizes the imaging devices 130, 130′, 130″. The database of structured reports may be compiled in a variety of manners. In a first example, a repository database that may be local or remote may be used to store all previously generated reports from analyses of images generated by the imaging devices 130, 130′, 130″. The repository database may be accessed via the communications network 405. It should be noted that the rule generating device 410 may receive the database of structured reports and store it in the memory arrangement 510 or may remotely access the reports. In a second example, the rule generating device 410 may be installed at the institution and may receive the reports for a predetermined amount of time prior to using the rule generating device 410.

The reports may be structured in that they are generated based upon FCs. Through FCs, the rule generating device 410 may be capable of extracting relevant information using the code component of the reports. The FCs may be standardized indicators of findings within images. Therefore, a structured report including a first FC from the imaging device 130 and a structured report including the first FC from the imaging device 130′ may correspond to the same type of finding within an image. Accordingly, the rule generating device 410 may be capable of categorizing these findings together.

However, it should be noted that the use of structured reports is only exemplary. Those skilled in the art will understand that reports may be “unstructured” when the FCs are not used. For example, a user or technician may create the reports with freehand. In another example, the user or technician may utilize different types of indicators that are not FCs. When unstructured reports are received, the rule generating device 410 may be configured with further applications used to normalize the content of the unstructured reports. For example, the normalizing may correspond the content to the appropriate FCs. Specifically, for discrete data points, a FC may be introduced to represent a situation in which the data point does not meet a predetermined clinically relevant threshold (e.g., ejection fraction of less than 50%). For relevant information described narratively, natural language processing engines may be used to automatically detect relevant information (e.g., smoking history). In this manner, an unstructured report may be analyzed in a substantially similar manner as structured reports by performing this additional step.

The candidate rule generating application 535 may utilize a plurality of logical templates such as “if . . . then . . . ” or exclusion principles. In this manner, the rule generating device 410 may determine a plurality of potential rules that may be included in the rule set. Each template may have different formats. For example, the if/then principle may also utilize the exclusion principle. In a particular format, the candidate rule generating application 535 may indicate that when any or all of a set of FCs is included in the report, then any or all of a further set of FCs are to be included or excluded. Again, this may be based upon the reports that were received by the rule generating device 410. The candidate rule generating application 535 may also utilize the vocabulary of the structured report database such as the FCs. Thus, the candidate rule generating application 535 may create a candidate rule by inserting one or more FCs in a logical template to derive the potential rules to be included in the rule set. For example, in the example described above, when a first FC indicates that the left ventricle is normal, and a second FC indicates that the left ventricle is severely dilated, the candidate rule may state that inclusion of the first and second FCs in a single report is incorrect or may prevent this scenario from occurring. That is, if the report is already to include the first FC, the user may be incapable of entering the second FC unless the first FC is handled previous to this selection.

It should be noted that the candidate rule generating application 535 may be configured to limit the number of candidate rules. Those skilled in the art will understand that the number of candidate rules that may be generated may grow exponentially with the number of FCs, the logical templates, etc. Thus, the candidate rule generating application 535 may use at least one heuristic to limit the number of candidate rules. In a first example, the candidate rule generating application 535 may be limited by not filling in more than N FCs in a list of FCs. In a second example, the candidate rule generating application 535 may not insert FCs that are relatively infrequent in the reporting database such as appearing less than a predetermined percentage of the reports. In a third example, the candidate rule generating application 535 may not insert combinations of FCs in a list of FCs that are relatively infrequent in the reporting database, again, such as appearing less than a predetermined percentage of the reports. The candidate rule generating application 535 may also utilize further heuristics to maintain a number of candidate rules for subsequent processing.

The rule scoring application 540 may accept the candidate rules generated by the candidate rule generating application 535 to determine one or more features that characterize the rule. Specifically, each feature may describe one aspect of the given candidate rule in machine-interpretable format. Thus, the rule scoring application 540 may include a feature generation subengine. The features may be based upon methods and techniques from various fields such as decision theory, statistics, clinical, natural language processing, spatial modeling, etc. The rule scoring application 540 may also include a scoring subengine that incorporates the various scores for each of the features to generate an overall score for the candidate rule.

In a first example, each candidate rule may be evaluated based upon decision theory. Specifically, whenever a candidate rule fires and detects an error, the rule may be awarded a positive preset score whereas when the rule fires in vein (i.e., false alarm), the rule may be debited a negative preset score. It is noted that the likelihood of a true positive and false positive occurring may be estimated from the database of received reports. Therefore, the utility of the candidate rule may be determined. A negative utility indicates that the candidate rule benefits are outweighed by the number of false alarms. For example, if a candidate rule states that FC-A and FC-B are not to appear together where FC-A is a first FC and FC-B is a second FC, the likelihood of a true positive may be estimated as P(!B|A)=(#(A&!B)/#A) and the likelihood of a false positive may be estimated as P(B|A)=(#(A&B)/#A). Accordingly, by decision theoretic measures, the utility of the rule may be X×P(!B|A)−Y×P(B|A). This calculation may be used by the rule scoring application 540 to determine a value for this characteristic relating to the candidate rule being evaluated.

In a second example, each candidate rule may be evaluated based upon statistics. Specifically, valid rules may detect combinations of FCs that are relatively infrequent in the report database. The relative frequency of combinations of FCs may be determined through statistical methods. For example, for the candidate rule described above where FC-A and FC-B are not to appear together, the rule scoring application 540 has an expectation to see relatively few reports that have both FC-A and FC-B. However, if this combination of FCs is common, there is also an expectation that that these FCs do not exclude each other. One mechanism of assessing relative infrequency is through comparison of the expected frequency based upon prior probabilities against the observed frequency. The prior probability of FC-A appearing may be estimated as P(A)=(#A/N) while the prior probability of FC-B appearing may be estimated as P(B)=(#B/N). If FC-A and FC-B are statistically independent, the rule scoring application 540 may expect to see P(A)×P(B)×N reports with the combination of FC-A and FC-B. This quantity may be compared against an observed number of reports including both FC-A and FC-B using #(A&B)/P(A)×P(B)×N. This comparison may be formalized by a ratio denoted as P(A&B)/P(A)P(B), P(A&B)−P(A)P(B). More advanced statistical methods may be used to compute a likelihood (e.g., p value) that FC-A and FC-B are statistically dependent (e.g., χ²) or that the number of observed FC-A and FC-B is a result of mis-clicks (modeled as random noise processes). This calculation may be used by the rule scoring application 540 to determine a value for this characteristic relating to the candidate rule being evaluated.

In a third example, each candidate rule may be evaluated by clinical aspects. Specifically, the clinical aspects may relate to disruptions in workflow from violation detections. The clinical feature may return the portion of reports on which the candidate rule would fire. If this portion of reports is too high, this may be a reason for the rule scoring application 540 to ignore the candidate rule. This calculation may be used by the rule scoring application 540 to determine a value for this characteristic relating to the candidate rule being evaluated.

In a fourth example, each candidate rule may be evaluated by natural language processing aspects. Specifically, the narrative component of the candidate rule may be used for matching. Through a list of devised keywords (e.g., normal, mild, moderate, severe, etc.) that may appear in the narrative components of the FCs (which may be stored in a separate keywords database), the rule scoring application 540 may compare its appearance against the logical template and a remainder of the narrative components. For example, the natural language processing may approve a candidate rule if keywords (e.g., normal and severe) are combined in a logical template such as “if left ventricle is normal, then left ventricle is not severely dilated.” This calculation may be used by the rule scoring application 540 to determine a value for this characteristic relating to the candidate rule being evaluated.

In a fifth example, each candidate rule may be evaluated by spatial modeling aspects. Specifically, a spatial model may be devised of the cardiac anatomy including the main anatomical entities (e.g., ventricles, atriums, etc.) and their spatial (e.g., “is connected to”) and functional (e.g., “is downstream from”) relationships. This spatial information may be stored in a separate spatial model database. For example, the list of anatomical locations may include the vena cava, the right atrium, the tricuspid valve, the right ventricle, the pulmonary valve, the left atrium, the mitral valve, the left ventricle, the aortic valve and coronary cusp, the aorta, the atrial (left and right atrium), the ventricular (left and right ventricle), and valvular (all valves). A list of keywords may be devised to detect anatomical entities for each of these anatomical locations. The spatial modeling aspects may return only candidate rules that contain FCs whose detected anatomical entities are identical or are nearby. For example, a distance matrix may be used to model the distance between two anatomical locations. In this manner, the distance between the vena cava and right atrium may be a first value (e.g., “1”) while the distance between the vena cava and the tricuspid valve may be a second value (e.g., “2”). This calculation may be used by the rule scoring application 540 to determine a value for this characteristic relating to the candidate rule being evaluated.

Once the scores for each of the features is determined, the rule scoring application 540 via the scoring subengine may determine the overall score of the candidate rule. The overall score may be indicative of whether the candidate rule is to be adopted in the rule set. This decision process may be modeled as a decision rule, a decision tree, a statistical or machine learning classifier (e.g., random forest, neural network, support vector machine, etc.), etc. In a first example, the statistical and machine learning classifiers may require training data in which sample rules may be used by a domain expert in an offline workflow. Assuming that the sample rules are to be adopted by the rule scoring application 540, feature thresholds and/or calibration of classifiers may be used to ultimately determine whether the candidate rule is to be adopted. In a second example, the rule scoring application 540 may return a numerical value that reflects a certainty that the given candidate rule is to be adopted. The candidate rule may be sorted by certainty value and a decision to adopt rules above a predetermined threshold value may be used. Other criteria for adoption of the rules in the rule set may include using only the top N rules as a meta-inclusion rule. It should be noted that a predetermined weighting factor may be applied to the scores (e.g., a first feature may be viewed as more important than a second feature) in generating the numerical value of the overall score.

The interface application 545 may persist the assessed candidate rules in human and/or machine interpretable format using standard serialization and writing mechanisms. Furthermore, the interface application 545 may be configured to include the certainty values of the assessed candidate rules and/or to include all the candidate rules for human review.

It should be noted that the rule adoption feature described above is only exemplary. When the scoring subengine has generated the overall score for each of the candidate rules, the set of candidate rules with their accompanying score may be presented to the user via the display device 515. The user may then manually determine which candidate rules are to be adopted into the rule set. Accordingly, the processor 505 may also execute a rule review application (not shown) that consumes the persisted selection of candidate rules and displays them in a convenient and intuitive manner to the user. The user may then be able to modify the rule assessments (e.g., to overrule the assessment of the rule scoring application 540). A domain expert may also use this application to verify desirability of the rules in the institution. The application may persist modified rule sets through the interface application 545.

The rule execution application 550 enables the adopted candidate rules to be included in the rule set to be used for the institution including the imaging devices 130, 130′, 130″. As the rule set is tailored based upon the reports generated by these imaging devices 130, 130′, 130″, the rule set is specifically designed to determine when contradictions or otherwise unlikely combinations of FCs exist. The imaging devices 130, 130′, 130″ may be loaded with the rule set. Therefore, when a user is utilizing the imaging device 130 and begins an analysis of an image by entering findings via the reporting pane 300, any violation of a rule may generate an appropriate action to be performed. For example, if the user enters a first FC and subsequently enters a second FC and the combination of these FCs results in a violation of the rule, the imaging device 130 may generate an alert to indicate to the user that there is this violation. In another example, if the user enters a first FC and subsequently completes the process of reporting but the rules indicate that a second FC is to be included when the first FC is entered, the imaging device 130 may generate a suggestion to include the second FC.

It should be noted that the processor 505 may further execute an in-workflow feedback collection application (not shown) upon a rule set being implemented at an institution. While using the automatically generated rule set using the above described mechanism, a tool may be configured with user feedback options. In this manner, the user may indicate that a detected violation is useful (e.g., “very helpful”) or not (e.g., “useless”). A voting mechanism may be implemented by which a rule is disabled if a predetermined number of negative votes is exceeded or if a predetermined percentage of votes is negative.

FIG. 6 shows a method 600 of generating a rule set according to the exemplary embodiments. The method 600 may be determined by the rule generating device 410 that may have access to reports generated by a plurality of imaging devices such as the imaging devices 130, 130′, 130″ via the communications network 405. The method 600 will be described with reference to these components.

In step 605, the rule generating device 410 receives previously generated reports from imaging devices of the institution such as the imaging devices 130, 130′, 130″. As discussed above, the reports may be received in a variety of manners. In a first example, the rule generating device 410 may be incorporated into the network 400 such that the imaging devices 130, 130′, 130″ may transmit reports to the rule generating device 410 via the communications network 405. In a second example, the rule generating device 410 may receive the reports in an offline manner in which a user may manually load the reports to be received on the rule generating device 410.

In step 610, the rule generating device 410 may normalize the reports. As discussed above, the reports may be structured or unstructured. When structured, the rule generating device 410 may extract the FCs in the reports. When unstructured, the rule generating device 410 may utilize various engines and processors (e.g., natural language processor) to extract the content of the reports into corresponding FCs or generate new FCs for the information in the reports.

In step 615, the candidate rule generating application 535 of the rule generating device 410 may generate candidate rules based upon the information of the reports. As discussed above, the candidate rules may be generated based upon templates including if/then, inclusion, exclusion, etc. formats. It should be noted that the method 600 may include further steps such as one where the candidate rule generating application 535 determines whether the number of candidate rules are within predetermined parameters such as those identified with heuristics.

In step 620, the rule generating application 410 such as through a feature generating subengine may generate feature values for each rule. The feature values may include aspects that characterize the rule in a machine-interpretable format. The features may be based on methods and techniques from various fields such as those listed above including decision theory, statistics, clinical aspects, natural language processing aspects, and spatial modeling aspects.

In step 625, the rule scoring application 540 of the rule generating application 410 may generate a respective score for each feature of the candidate rule. The scores may be determined for each score using a respective calculation corresponding to the type of feature characteristic. The rule scoring application 540 may also normalize the scores of the different features to subsequently determine an overall score for the feature.

In step 630, the rule generating device 410 may determine whether a candidate rule is adopted based upon the scores for each feature or based upon the overall score for the feature. For example, when the rule generating device 410 utilizes each feature as a determinant, a corresponding predetermined threshold may be used as a basis. In another example, when the rule generating device 410 utilizes an overall score, a predetermined threshold may also be used as a basis. In this manner, the rule generating device 410 may automatically determine which candidate rules are to be adopted into the rule set.

It should again be noted that the method 600 may include additional steps such as if the number of candidate rules to be adopted is not to exceed a predetermined number. When such a step is used, the method 600 may include further substeps such as readjusting the threshold value used in step 630 to filter the candidate rules that are adopted.

In step 635, the rule generating device 410 determines whether a candidate rule is adopted into the rule set for the institution. Again, this may relate to the rule generating device 410 performing an automatic determination of whether the candidate rule is to be adopted. If the rule is to be adopted, the rule generating device 410 may continue the method to step 640. In step 640, a user may provide an override via the interface application 545 where a candidate rule that is to be adopted is eliminated instead. Thus, if the override is performed, the rule generating device 410 continues the method to step 645. However, if the user does not override the candidate rule from being adopted, the rule generating device 410 continues the method 600 to step 650 where the candidate rule is added to the rule set. Returning to step 635, if the candidate rule is determined to not be adopted, the rule generating device 410 continues the method 600 to step 655. In step 655, a substantially similar override step may be performed in which the user may override the determination to eliminate the candidate rule. Thus, if the user accepts the decision to not adopt the rule, the rule generating device 410 continues the method to step 545. However, if the user overrides the determination of the rule generating device 410, the rule generating device 410 continues the method 600 to step 650 where the candidate rule is added to the rule set.

In step 660, the rule generating device 410 determines whether there are any further candidate rules to be evaluated for adoption. If there are further candidate rules, the rule generating device 410 returns the method 600 to step 630. However, if all candidate rules have been evaluated for adoption, the rule generating device 410 continues the method 600 to step 665 where the rule set is generated for the institution and the rule execution application 550 configures the imaging devices 130, 130′, 130″ to provide quality assurance of report generation based upon the rule set.

It should again be noted that the method 600 may include further steps upon the rule set being implemented. For example, an in-work flow feedback collection application of the rule generating device 410 may be used to determine how the rule set is performing subsequent to implementation. Specifically, the feedback application may receive inputs from the user of the imaging device 130 that prompts for a response to whether a rule was helpful. When a predetermined number of responses are received, the feedback application may determine whether to update the rule set to potentially include further rules or eliminate existing rules.

According to the exemplary embodiments, the system and method of the exemplary embodiments provide a mechanism to automatically determine quality assurance rules in echocardiogram interpretation workflows. Specifically, the rules may ensure that contradictions or incompatible statements in a report are prevented from being shown on the report. By first receiving previously generated reports from a plurality of imaging devices of an institution, a rule generating device may first generate candidate rules and evaluate the candidate rules based upon scores of features that characterize the candidate rule. Subsequently, the rule generating device may automatically determine whether the candidate rule is to be adopted into a rule set implemented for the imaging devices of the institution (subject to user intervention). In this manner, the institution may overcome a lack of expert resources that would normally be required to manually create the rules. The capability of generating rule sets for different institutions is also capable, thereby overcoming the inability to port rules.

Those skilled in the art will understand that the above described exemplary embodiments may be implemented in any suitable software or hardware configuration or combination thereof. An exemplary hardware platform for implementing the exemplary embodiments may include, for example, an Intel x86 based platform with compatible operating system, a MAC platform and MAC OS, a mobile hardware device having an operating system such as iOS, Android, etc. In a further example, the exemplary embodiments of the above described method may be embodied as a program containing lines of code stored on a non-transitory computer readable storage medium that, when compiled, may be executed on a processor or microprocessor.

It will be apparent to those skilled in the art that various modifications may be made in the present invention, without departing from the spirit or the scope of the invention. Thus, it is intended that the present invention cover modifications and variations of this invention provided they come within the scope of the appended claims and their equivalent.

1. A method, comprising:
receiving, by a rule generating device, a plurality of previously generated reports, each of the previously generated reports including respective analysis content of a respective image;
generating, by the rule generating device, a candidate rule based upon the analysis content, the candidate rule configured to increase a quality assurance of future reports;
generating, by the rule generating device, a respective score for each candidate rule based upon the candidate rule and the previously generated reports; and
including, by the rule generating device, the candidate rule into a rule set when the score is above a predetermined threshold.

2. The method of claim 1, further comprising:
determining, by the rule generating device, at least one feature characterizing the candidate rule based upon predetermined criteria that the candidate rule satisfies for the quality assurance.

3. The method of claim 1, wherein the previously generated reports are at least one of structured reports where the analysis content is defined with predetermined indicators and unstructured reports where the analysis content includes natural language.

4. The method of claim 3, further comprising:
normalizing, by the rule generating device, the unstructured reports by determining the natural language to corresponding select ones of the predetermined indicators.

5. The method of claim 1, wherein the candidate rule is formatted in a template based upon one of an inclusion, an exclusion, and a combination thereof.

6. The method of claim 1, wherein the features include at least one of decision theory, statistics, clinical aspects, natural language processing aspects, and spatial modeling aspects.

7. The method of claim 6, wherein the score is an overall score generated based upon individual subscores for each of the features.

8. The method of claim 6, wherein the score includes a plurality of scores respective for each of the features.

9. The method of claim 8, further comprising:
evaluating each of the plurality of scores with a respective predetermined threshold.

10. The method of claim 1, wherein image is generated by an echocardiogram procedure.

11. A device, comprising:
a receiver configured to receive a plurality of previously generated reports, each of the previously generated reports including respective analysis content of a respective image; and
a processor configured to a rule set where each rule is configured to increase a quality assurance of future reports,
wherein the processor is further configured to:
generate a candidate rule based upon the analysis content, the candidate rule configured to increase a quality assurance of future reports;
generate a respective score for each candidate rule based upon the candidate rule and the previously generated reports; and
include the candidate rule into the rule set when the score is above a predetermined threshold.

12. The device of claim 11, wherein the processor is further configured to determine at least one feature characterizing the candidate rule based upon predetermined criteria that the candidate rule satisfies for the quality assurance.

13. The device of claim 11, wherein the previously generated reports are at least one of structured reports where the analysis content is defined with predetermined indicators and unstructured reports where the analysis content includes natural language.

14. The device of claim 13, wherein the processor is further configured to normalize the unstructured reports by determining the natural language to corresponding select ones of the predetermined indicators.

15. (canceled)

16. (canceled)

17. (canceled)

18. (canceled)

19. (canceled)

20. A system, comprising:
a first imaging device configured to generate a first image for a first analysis, a first report being generated based upon the first analysis;
at least one second imaging device configured to generate a respective second image for a second analysis, a respective second report being generated based upon the second analysis;
a rule generating device configured to receive the first and second reports, the rule generating device configured to generate a rule set including a plurality of rules configured to increase a quality assurance of future reports generated by the first and second imaging devices, the rule set being generated by:
generating a candidate rule based upon the first and second analyses;
generating a respective score for each candidate rule based upon the candidate rule and the first and second reports; and
include the candidate rule into the rule set when the score is above a predetermined threshold.
